(19) United States
(12) Patent Application Publication
(10) Pub. No.: US 2018/0264155 A1
(43) Pub. Date: Sep. 20, 2018

(54) FLEXIBLE STERILIZATION DIVIDER

(71) Applicant: Summit Medical, Inc, St. Paul, MN (US)

(72) Inventors: Jacob Kramer, Farmington, MN (US); Daniel Broberg, Deephaven, MN (US)

(21) Appl. No.: 15/920,653

(22) Filed: Mar. 14, 2018

Related U.S. Application Data
(60) Provisional application No. 62/471,527, filed on Mar. 15, 2017.

(51) Int. Cl. A61L 2/26; A61B 50/33

(52) U.S. Cl. CPC A61L 2/26; A61B 2050/3008; A61B 50/33

(57) ABSTRACT

Disclosed herein are various embodiments of sterilization tray dividers and related devices, systems and methods. In one embodiment, a sterilization tray divider comprises a divider wall comprising a flexible material able to withstand medical sterilization processes and chemicals.

### RELATED APPLICATION

The present application claims the benefit of U.S. Provisional Application No. 62/471,527 filed Mar. 15, 2017, which is hereby incorporated herein in its entirety by reference.

### TECHNICAL FIELD

Embodiments relate to sterilization of medical equipment, and, more particularly, to sterilization trays and related devices used for sterilization of medical equipment.

### BACKGROUND

Sterilization trays are used in the medical and dental industries to provide storage and organization for surgical and dental instruments and other medical devices that require sterilization. Sterilization trays must provide organizational structures within the sterilization trays that not only ensure that each instrument is secured but that the overall placement of the instruments within each surgical tray provides an adequate fluidic flow path for sterilant (which may be a liquid or a gas) to adequately access and sterilize each instrument during sterilization processes.

To meet these requirements, conventional sterilization trays are constructed using rigid, medical grade materials (e.g., medical grade stainless steel, aluminum and plastics) that can withstand both autoclave and chemical sterilization, and are generally rectangular boxes having a base with four adjacent walls and a removable top. To promote sterilant flow through the sterilization tray, the bottom, walls, and top generally contain a plurality of sterilant flow apertures. Further, conventional sterilization trays include a plurality of fixed dividers that secure, separate, and organize the plurality of surgical instruments within the surgical tray.

Conventionally, these dividers are made from the same rigid material that the surgical tray is made from and are custom-designed for the type of surgical instrument to be held in the tray. These dividers are either fixedly attached or removably coupled to the sterilization tray either using the existing sterilant flow apertures or other fixation means.

Because of the large and ever-increasing number and form factor of instruments to be sterilized, the dividers and trays must be custom built to accommodate each type of instrument. This level of customization increases cost and complexity.

To address this problem, the surgical tray industry has focused on creating simple divider walls that can be used in a variety of surgical tray platforms and with a variety of devices to be sterilized. These simple dividers, however, often are not sufficiently customizable and suffer from other drawbacks.

For example, many advanced surgical instruments have electrical and communication cables, and these cables must also be sterilized with the instrument (i.e., the cables often are fixedly coupled to the instrument) in a sterilization tray. Conventionally, the cables must be wrapped around surgical divider walls with fixed and prefabricated curvatures to ensure that the cable is secure and properly arranged for sterilant flow. Different cables, however, may require different wrap radii, given the diameter, flexibility and type (i.e., an instrument cable configured to rout electrical power only will have a different acceptable wrap radius when compared to a cable configured to rout electrical power, fiber optics and communicative wiring) of cable. In one particular example, some scopes have cables that contain fiber optics. These cables have a minimum bend radius and cannot be wrapped like other (e.g., simple electrical) cables because tight bend radii can cause damage to the fiber optics. The industry has addressed this problem by creating multiple curved dividers with differing radii, but this again introduces complexity and requires custom trays and dividers to be designed and assembled.

### SUMMARY

Disclosed herein are various embodiments of flexible sterilization tray dividers. In embodiments, a sterilization tray divider comprises a divider wall, the divider wall comprising a flexible material configured to withstand medical sterilization.

The divider wall comprises a first side, a second side opposite the first side, and a mounting surface configured to interface with a surgical tray to which the sterilization tray divider can be selectively and removable coupled. The mounting surface comprises at least one fixation element configured to selectively and removable couple the divider to the sterilization tray.

In one embodiment, at least one fixation element comprises a flexible peg. The flexible peg can be integrally formed with or coupled to the mounting surface at a first end of the peg and can comprise a flexible flared second end, such that the flexible flared second end can temporarily deform in order to pass through an aperture in surgical tray and then expand back to its flared state in order to secure the peg, and therefore the sterilization divider, to the sterilization tray.

In another embodiment, the fixation element comprises a female threaded aperture. The threaded aperture can comprise metal (e.g., stainless steel) and can be recessed in the mounting surface and fixedly coupled to the sterilization divider. A correspondingly threaded male fixation element can interface with the female threaded aperture by passing through an aperture in a sterilization tray from an opposing side to selectively and removably couple the sterilization divider to the tray. In other embodiments, the male and female arrangements of the fixation elements can be reversed and/or other types of fixation elements (e.g., male and female snap-fit elements) can be used in other embodiments.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A is a front view of a surgical tray divider according to an embodiment.

FIG. 1B is a side view of a surgical tray divider according to an embodiment.

FIG. 1C is a bottom view of a surgical tray divider according to an embodiment.

FIG. 2A is a front view of a surgical tray divider according to an embodiment.

FIG. 2B is a side view of a surgical tray divider according to an embodiment.

FIG. 3 is a top view of a plurality of surgical tray dividers arranged on a surgical tray bottom according to an embodiment.

FIG. 4 is a front view of a surgical tray divider coupled to a surgical tray bottom according to an embodiment.

FIG. 5 is a front view of a surgical tray divider coupled to a surgical tray bottom according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Described herein are embodiments of sterilization tray dividers and related devices, systems and methods. Specifically, flexible sterilization tray dividers for use with sterilization trays are disclosed. Flexible sterilization dividers provide added versatility and increased instrument protection when compared with conventional rigid sterilization dividers.

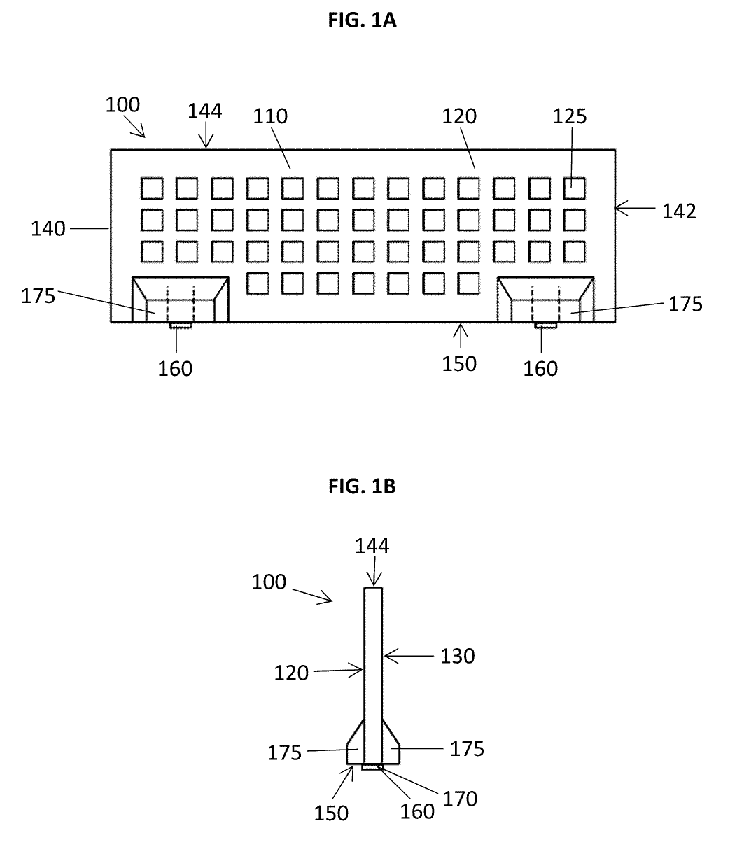

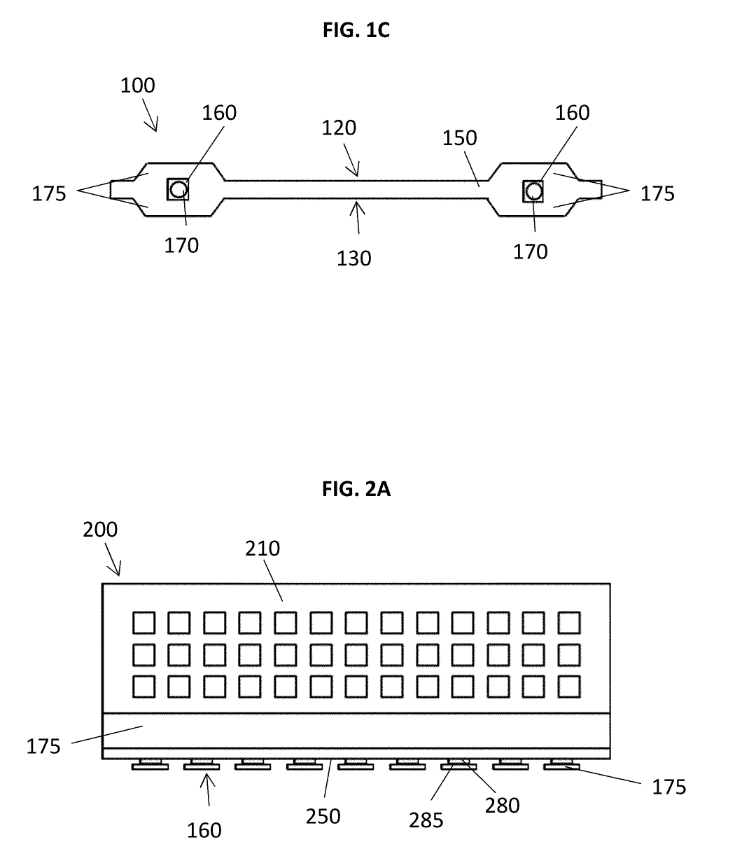

Referring now to FIGS. 1A and 1B, an embodiment of a flexible sterilization tray divider 100 is shown. Divider 100 can be flexed into a variety of shapes and configurations, and mounted to a sterilization tray in those various configurations, as discussed below in more detail.

Flexible sterilization divider 100 includes a wall 110 having a first side 120 and a second side 130 opposite the first side 120. In an embodiment, wall 110 includes at least one aperture 125 passing through wall 110 from first side 120 to second side 130 to create a void therethrough. As depicted in FIG. 1A, wall 110 comprises an array of apertures 125 spaced apart from one another. Apertures 125 allow for sterilant to flow through, rather than around, flexible sterilization divider 100. In other embodiments, wall 110 can comprise variously shaped, sized and arranged apertures 125, such as to accommodate sufficient sterilant flow with respect to a particular piece or type of medical equipment to be sterilized. For example, in some embodiments, wall 110 comprises one or more larger apertures 125 proximate or interrupting the bottom surface (e.g., mounting surface 150 discussed below) in order to assist with or improve flow, drainage, or other movement of sterilant fluid.

In other embodiments, wall 110 could also have no apertures 125 and instead be formed of a solid or substantially solid piece of flexible material. In such an embodiment, wall 110 can be used for cushioning or protection of a medical device to be sterilized, such as by being placed along or against a wall of a sterilization tray so that the medical device does not come into contact with the (typically rigid and metal) material of the tray.

In these and other embodiments, wall 110 can comprise one or more spacers, projections or other features (not shown) extending from first side 120 and/or second side 130 in order to provide a space for sterilant flow between wall 100 and a wall or other component of or in the sterilization tray. Spacers can be provided for other uses (e.g., to anchor or hold cables or other devices in place during sterilization, to provide a minimum separation between wraps of cable or other components, to selectively and removably couple first side 120 or second side 130 to a sterilization tray, another divider 100, or some other component, etc.) or in other embodiments (e.g., embodiments comprising apertures 125). In these embodiments, the spacers can have a variety of configurations, such as J-, C-, or U-shaped hooks; rounded or other-shaped nubs; flat or rounded projections; or some other configuration.

In embodiments, wall 110 is defined by a first edge 140, a second edge 142, a third edge 144 and a fourth edge, a mounting surface 150, which are arranged orthogonal to first side 120 and second side 130. In some embodiments, first edge 140 and third edge 144 meet at a substantially squared corner, while in other embodiments first edge 140 and third edge 144 meet at a substantially rounded corner, other shape, and/or other angle (e.g., an angle other than substantially 90 degrees). In some embodiments, second edge 142 and third edge 144 meet at a substantially squared corner, while in other embodiments second edge 142 and third edge 144 meet at a substantially rounded corner, other shape, and/or other angle (e.g., an angle other than substantially 90 degrees). In some embodiments, first edge 140 and mounting surface 150 meet at a substantially squared corner, while in other embodiments first edge 140 and mounting surface 150 meet at a substantially rounded corner, other shape, and/or other angle (e.g., an angle other than substantially 90 degrees). In some embodiments, second edge 142 and mounting surface 150 meet at a substantially squared corner, while in other embodiments, second edge 142 and mounting surface 150 meet at a substantially rounded corner, other shape, and/or or other angle (e.g., an angle other than substantially 90 degrees).

In embodiments, mounting surface 150 comprises one or more features configured to selectively and removably couple divider 100 to various sterilization trays. In embodiments, these features comprise one or more fixation elements 160 arranged on mounting surface 150. Fixation elements 160 can have a variety of forms and features, and some examples will be discussed herein. Those skilled in the art, however, will recognize that other fixation elements 160 or approaches can be used in various other embodiments. For example, in some embodiments fixation elements 160 may be selected, sized, shaped or other configured for use with a particular sterilization tray, which may comprise apertures or other elements of a unique or non-standard size.

In an embodiment shown in FIG. 1C, divider 100 comprises two fixation elements 160, which comprise female threaded elements 170. Elements 170 are configured to interact with correspondingly threaded male fixation elements (not shown) to selectively and removable couple divider 100 to a sterilization tray. For example, divider 100 can be arranged in the tray with elements 170 aligned with apertures in the tray. Corresponding male elements are then inserted into elements 170 from outside the tray, such that the tray is captured between the male elements and divider 100, thereby securing divider 100 to the tray.

In embodiments, female element 170 comprises a material suitable for threaded fixation, such as medical grade stainless steel, aluminum, or other materials. As depicted in FIG. 1C, female elements 170 are recessed in wall 100 from mounting surface 150. In other embodiments, female element 170 is coupled to lower mounting face 150 via over-molding, adhesive, or some other suitable fixation method. In other embodiments not shown here, the male and female features of the fixation system can be reversed, such that fixation elements 160 comprise male inserts having external threading or other suitable fixation devices to interact with a corresponding female fixation element coupled therewith from an opposing (e.g., external) surface of the tray.

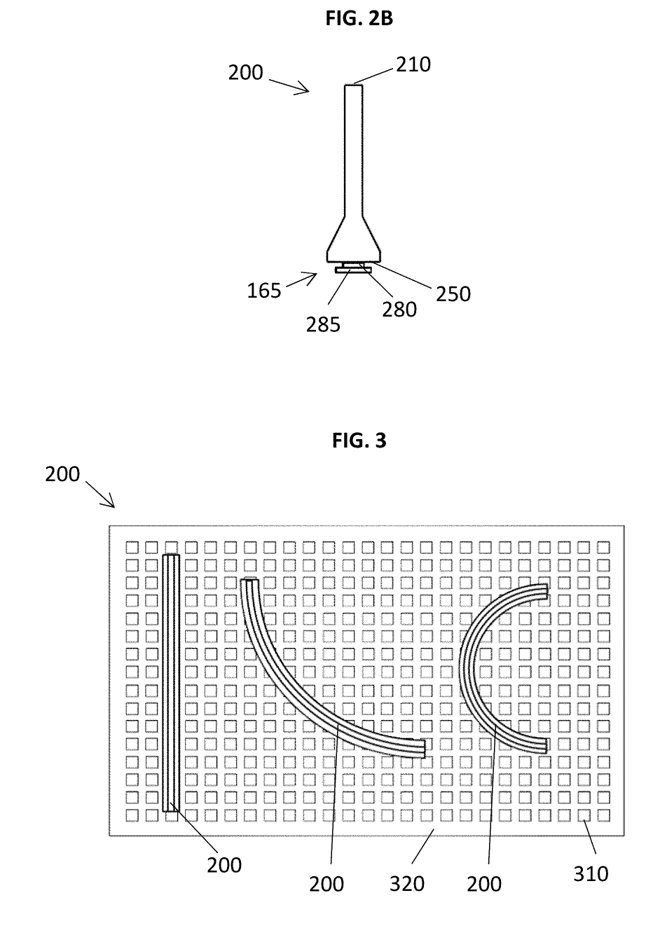

In some embodiments, wall 110 comprises reinforced areas to accommodate fixation elements 160, such as support flares 175 arranged on first side 120 and second side 130 and adjacent to fixation features 160 in FIG. 1C. In other embodiments, support flares 175 can span the entire lower portion of first side 120 and second side 130, as an embodiment shows in FIG. 2A-B, such that wall 110 is thicker along mounting surface 150 than along third edge 144. In still other embodiments, the portion of wall 110 proximate mounting surface and/or other portions of wall 110 can vary in thickness, material, shape or other configuration, such as to accommodate a particular piece of equipment to be sterilized, a unique sterilization tray, or some other element.

Referring now to FIG. 2A-B, in another alternative embodiment a flexible sterilization divider 200 includes fixation elements 160 comprising pegs 280 that extend, at a first end of each peg 280, from mounting surface 250. A second end of each peg 280 is coupled to or comprises a flexible extension 285. In embodiments, peg 280 is flexible, and both peg 280 and extension 285 are configured to sufficiently deform such that extension 285 can be passed through an aperture in a sterilization tray and re-form thereafter to selectively and removably couple divider 200 to the tray. In embodiments, wall 210, pegs 280 and extensions 285 can be unitarily formed and comprise the same material. In other embodiments, pegs 280 and extensions 285 can be fixedly coupled with wall 210.

In embodiments, dividers 100 and 200 comprise a sufficiently flexible, though also sufficiently rigid, material capable of withstanding medical sterilization procedures. For example, dividers 100 and 200 can comprise a flexible elastomer, such as thermoplastic elastomer (TPE), or thermoplastic rubber in various embodiments. In a particular embodiment, dividers 100 and 200 comprise silicone. Flexible elastomers and silicone can be sufficiently flexible so as to enable dividers 100 and 200 to be positioned and repositioned in a variety of configurations (discussed in more detail below) while also be sufficiently rigid so as to maintain shape and keep cables, devices and other equipment to be sterilized in place during sterilization and/or storage in the tray. In embodiments, the material used for dividers 100 and 200 has a durometer in a range of about 30 to about 100 on the Shore A scale, for example a durometer of about 60 to about 90, such as a durometer of about 80 in one example embodiment. Lower durometers (i.e., softer materials) can be used in embodiments in which dividers 100 or 200 are being used for shielding or cushioning (e.g., against a wall of a sterilization tray divider); in such embodiments, a durometer of about 30 can be suitable. Higher durometers (e.g., harder materials) can be used in embodiments in which dividers 100 or 200 are desired to maintain a particular shape to hold equipment within a tray; in such embodiments, a durometer of about 80 can be suitable. Certain applications or embodiments may necessitate for durometers that are greater or lesser than these examples. In other embodiments, dividers 100 and 200 comprise some other suitable polymer, plastic, rubber, or other material having sufficient rigidity, flexibility and durability in medical sterilization environments. In still other embodiments, other materials can be embedded within or otherwise included in dividers 100 and 200 to provide sufficient flexibility and/or shape retention. For example, in some embodiments a flexible metal wire or other material can be embedded within divider 100 or 200, providing some additional structure to the divider so that it can generally hold a shape though still can be reshaped as needed or desired.

Referring now to FIG. 3, three flexible sterilization dividers 200 are shown secured to a sterilization tray 300 in various orientations. Because of the flexibility of the material of flexible sterilization dividers 200, the same dividers 200 are capable of conforming to any number of orientations bound only by the pattern and spacing of sterilization tray apertures 310 arranged on sterilization tray bottom 320 and the arrangement of fixation elements (not visible in FIG. 3) on dividers 200. As shown in FIG. 3, flexible sterilization divider 200 is show in a substantially straight orientation, 90 degree arc, and a 180 degree arc as examples of the variety of orientations that could be created. Other configurations, including S-shapes, C-shapes, U-shapes, L-shapes, J-shapes, larger or smaller arcs, and straight lines, are among those that are possible, with a single divider capable of being configured and reconfigured into and between any of these shapes as needed or desired.

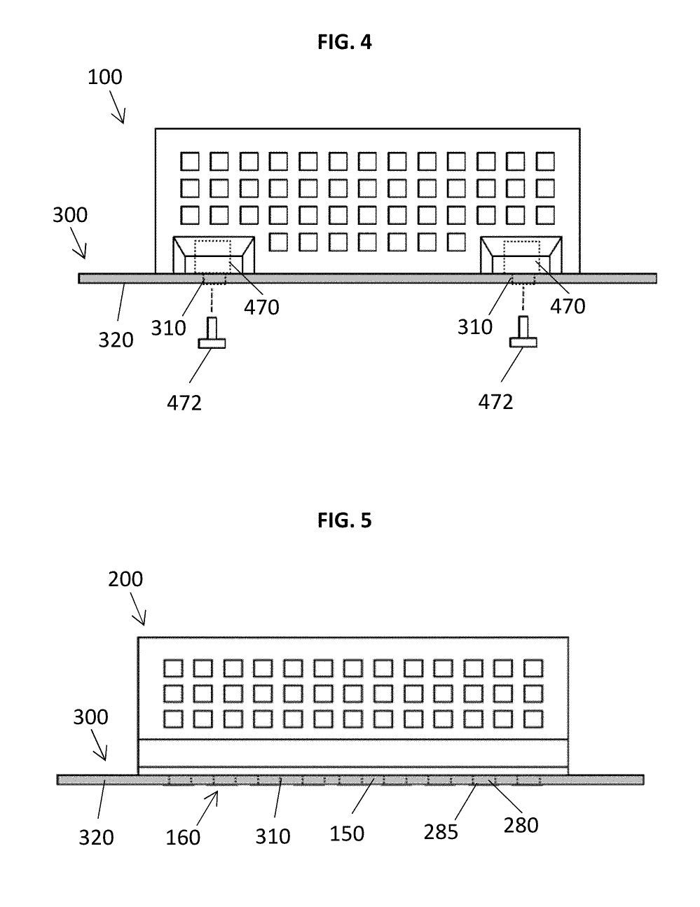

Referring now to FIG. 4, a side cross-sectional view of a divider 100 coupled to tray 300 in FIG. 3 is shown. In this example embodiment, female fixation element 470 is configured to threadingly engage male fixation elements (e.g., screws) 472. In use, a user may arrange flexible sterilization divider 100 in any configuration desired, arrange and align fixation elements 470 with corresponding sterilization tray apertures 310, and from the opposite side of sterilization tray bottom 320 insert screws 472 into each fixation element 470, thereby securing divider 100 to tray 300. Screw 472 can be easily removed and divider 100 reconfigured after sterilization or as needed or desired.

In another embodiment shown in FIG. 5, flexible sterilization divider 200 comprising pegs 280 and extensions 285 is shown coupled to wall 320 of tray 300. In this embodiment, pegs 280 extend through apertures 310 in wall 320, such that pegs 280 substantially remain within apertures 310 while extensions 285 re-expand after having temporarily deformed to pass through apertures 310 and now rest against the outer surface of bottom wall 320 to secure divider 200 to bottom wall 320. Extensions 285 can be re-deformed and passed back through apertures 310 to decouple divider 200 from tray 300 as desired.

Because of the flexibility of sterilization tray divider 100 and 200, one or more dividers 100 and/or 200 can be used with a single sterilization tray, configured and reconfigured in a variety of shapes to accommodate a wide range of surgical instruments and other devices to be sterilized. Users thus can create custom tray arrangements on the fly using sterilization equipment (e.g., dividers 100 and/or 200) on hand, avoiding the expense and time associated with having a tray manufacturer design and build a custom but dedicated use tray. This enables one divider 100 or 200 to be used in ways that conventionally required a plurality of inflexible dividers with fixed shapes. Moreover, the flexible material of the dividers can be easier on equipment and provide additional applications (e.g., cushioning or protection) not available with conventional rigid dividers. Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A sterilization tray divider for use with a sterilization tray comprising:
a divider wall comprising a flexible elastomer and having a first side and a second side opposing the first side, the first side and the second side being defined by a first edge, a second edge, a third edge and a mounting surface configured to oppose a first surface of a wall of the sterilization tray; and
at least one fixation element arranged on the mounting surface to selectively and removably couple the divider to the wall of the sterilization tray.

2. The sterilization tray divider of claim 1, wherein the flexible elastomer has a durometer in a range of about 30 to about 100 on the Shore A scale.

3. The sterilization tray divider of claim 2, wherein the flexible elastomer has a durometer in a range of about 50 to about 80 on the Shore A scale.

4. The sterilization tray divider of claim 1, wherein the flexible elastomer comprises silicone.

5. The sterilization tray divider of claim 1, further comprising at least one aperture extending through the divider wall from the first surface to the second surface.

6. The sterilization tray divider of claim 5, wherein the at least one aperture comprises an array of a plurality of apertures.

7. The sterilization tray divider of claim 1, wherein the at least one fixation element comprises a female connector configured to selectively and removably interact with a male connector arranged on a second surface of the wall of the sterilization tray, the second surface of the wall of the sterilization tray being opposite the first surface of the wall of the sterilization tray, with a portion of the male connector extending through an aperture in the wall of the sterilization tray to interact with the female connector.

8. The sterilization tray divider of claim 7, wherein the female connector and the male connector comprise correspondingly threaded connectors.

9. The sterilization tray divider of claim 1, wherein the fixation element comprises a peg configured to extend through an aperture in the wall of the sterilization tray from the first surface to a second surface opposite the first surface of the sterilization tray.

10. The sterilization tray divider of claim 9, wherein the peg is coupled at a first end with the mounting surface an at a second end with a extension configured to temporarily deform such that the extension passes through the aperture in the wall and to reform against the second surface of the sterilization tray.

11. The sterilization tray divider of claim 1, wherein the at least one fixation element comprises a male connector configured to selectively and removably interact with a female connector arranged on a second surface of the wall of the sterilization tray, the second surface of the wall of the sterilization tray being opposite the first surface of the wall of the sterilization tray, with a portion of the male connector extending through an aperture in the wall from the first surface to the second surface of the sterilization tray to interact with the female connector.

12. A sterilization tray system, comprising:
a sterilization tray comprising a bottom wall and at least one side wall, the bottom wall comprising a plurality of apertures extending through the bottom wall from a first surface of the bottom wall to a second surface of the bottom wall; and
at least one sterilization tray divider comprising
a divider wall comprising a flexible elastomer and having a first side and a second side opposing the first side, the first side and the second side being defined by a first edge, a second edge, a third edge and a mounting surface configured to oppose the first surface of the bottom wall of the sterilization tray, and
at least one fixation element arranged on the mounting surface to selectively and removably couple the divider to the bottom wall of the sterilization tray via at least one of the plurality of apertures.

13. The sterilization tray system of claim 12, wherein the flexible elastomer has a durometer in a range of about 30 to about 100 on the Shore A scale.

14. The sterilization tray system of claim 13, wherein the flexible elastomer has a durometer in a range of about 50 to about 80 on the Shore A scale.

15. The sterilization tray system of claim 12, wherein the flexible elastomer comprises silicone.

16. The sterilization tray system of claim 12, further comprising at least one aperture extending through the divider wall from the first surface of the divider wall to the second surface of the divider wall.

17. The sterilization tray system of claim 16, wherein the at least one aperture comprises an array of a plurality of apertures.

18. The sterilization tray system of claim 12, wherein the at least one fixation element comprises a female connector configured to selectively and removably interact with a male connector arranged on the second surface of the bottom wall of the sterilization tray, with a portion of the male connector extending through one of the plurality of apertures through the bottom wall of the sterilization tray to interact with the female connector.

19. The sterilization tray system of claim 18, wherein the female connector and the male connector comprise correspondingly threaded connectors.

20. The sterilization tray system of claim 12, wherein the fixation element comprises a peg configured to extend through one of the plurality of apertures in the bottom wall of the sterilization tray.

21. The sterilization tray system of claim 20, wherein the peg is coupled at a first end with the mounting surface and at a second end with a extension configured to temporarily deform such that the extension passes through the one of the plurality of apertures in the wall and to reform against the second surface of the bottom wall of the sterilization tray.

22. The sterilization tray system of claim 12, wherein the at least one fixation element comprises a male connector configured to selectively and removably interact with a female connector arranged on the second surface of the bottom wall of the sterilization tray, with a portion of the male connector extending through one of the plurality of apertures in the bottom wall of the sterilization tray to interact with the female connector.

23. The sterilization tray system of claim 12, wherein the divider wall is configured to be selectively and removably coupled to the bottom wall of the sterilization tray via the at least one fixation element in a plurality of different shapes or configurations.

24. A method for dividing sterilization trays comprising:
providing at least one sterilization tray divider comprising:
a divider wall comprising a flexible elastomer and having a first side and a second side opposing the first side, the first side and the second side being defined by a first edge, a second edge, a third edge and a mounting surface configured to oppose a first surface of a bottom wall of a sterilization tray, and
at least one first fixation element arranged on the mounting surface to selectively and removably couple the divider to the bottom wall of the sterilization tray via at least one of a plurality of apertures through the bottom wall.

25. The method of claim 24, further comprising:
providing at least one sterilization tray comprising the bottom wall and at least one side wall, the bottom wall comprising the plurality of apertures extending through the bottom wall from the first surface of the bottom wall to a second surface of the bottom wall.

26. The method of claim 25, further comprising:
providing at least one second fixation element configured to interact with the at least one first fixation element to selectively and removably couple the at least one sterilization tray divider to the at least one sterilization tray.

27. The method of claim 26, wherein the at least one first fixation element comprises a female connector configured to selectively and removably interact with the at least one second fixation element comprising a male connector, wherein the male connector is configured to be arranged on the second surface of the bottom wall of the sterilization tray, with a portion of the male connector extending through one of the plurality of apertures through the bottom wall of the sterilization tray to interact with the female connector.

28. The method of claim 26, wherein the at least one first fixation element comprises a male connector configured to selectively and removably interact with the at least one second fixation element comprising a female connector, wherein a portion of the male connector is configured to extend through one of the plurality of apertures through the bottom wall of the sterilization tray to interact with the female connector arranged against the second surface of the bottom wall.

29. The method of claim 25, further comprising coupling the at least one sterilization tray divider to the at least one sterilization tray in a first configuration.

30. The method of claim 29, further comprising:
removing the at least one sterilization tray divider from the at least one sterilization tray; and
recoupling the at least one sterilization tray divider to the at least one sterilization tray in a second configuration different from the first configuration.

31. The method of claim 24, further comprising forming at least one aperture extending through the divider wall from the first surface of the divider wall to the second surface of the divider wall.

32. The method of claim 31, wherein the at least one aperture comprises an array of a plurality of apertures.
